SEQUENCE LISTING (6 sequences)

SEQ ID NO: 1 - DNA, 30 bp, synthetic construct
  source 1..30: mol_type = other DNA
tttaccgcaactacacctaactgagatact

SEQ ID NO: 2 - DNA, 30 bp, synthetic construct
  source 1..30: mol_type = other DNA
ttagataaccggatacagtgactttgatag

SEQ ID NO: 3 - DNA, 30 bp, synthetic construct
  source 1..30: mol_type = other DNA
ctgcgtatggaggaaggaacttttgcgtgt

SEQ ID NO: 4 - DNA, 19 bp, synthetic construct
  source 1..19: mol_type = other DNA
gggttctctctgagtctgt

SEQ ID NO: 5 - RNA, 19 nt, synthetic construct
  source 1..19: mol_type = other RNA
aaaaaaaaaaaaaaaaaaa

SEQ ID NO: 6 - RNA, 14 nt, synthetic construct
  source 1..14: mol_type = other RNA
ctgatccaaggcta

(19) United States
(12) Patent Application Publication
(10) Pub. No.: US 2025/0376717 A1
(43) Pub. Date: Dec. 11, 2025

(54) METHOD OF ANALYSING RNA DEGRADATION

(71) Applicant: SINTEF TTO AS, Trondheim (NO)

(72) Inventor: Francis Combes, Trondheim (NO)

(21) Appl. No.: 18/876,817

(22) PCT Filed: Jun. 30, 2023
(86) PCT No.: PCT/EP2023/068125

§ 371 Date: Dec. 19, 2024

(30) Foreign Application Priority Data
Jul. 1, 2022 (GB) 2209732.3

(51) Int. Cl. C12Q 1/6825

(52) U.S. Cl. CPC C12Q 1/6825

(57) ABSTRACT

The present invention relates generally to methods of analysing ribonucleic acid (RNA). In particular, the invention relates to methods of analysing RNA to assess levels of degradation of RNA molecules in an RNA sample. The methods use an exonuclease and a means for detection of nucleotides and/or nucleosides. The invention provides a method of analysing ssRNA in a sample, said method comprising: (a) contacting the sample with a probe, wherein the probe binds to a target region of ssRNA in the sample; (b) incubating the sample with a single-strand specific exoribonuclease; and (c) detecting nucleotides and/or nucleosides generated by step (b), wherein the nucleotides and/or nucleosides are derived from one or more RNA regions located outside the target region of ssRNA in the sample. The invention also provides kits for performing said method.

The present invention relates generally to methods of analysing ribonucleic acid (RNA). In particular, the invention relates to methods of analysing RNA to assess levels of degradation of RNA molecules in an RNA sample. The methods use an exonuclease and a means for detection of nucleotides and/or nucleosides.

RNA is a polymeric molecule essential in various biological roles, including the expression of genes. It is thus highly desirable for biochemical researchers to be able to analyse specific RNA molecules of interest, and for medical practitioners to be able to use RNA molecules in RNA therapy, for example micro RNA (miRNA) and messenger RNA (mRNA).

However, RNA is relatively unstable and so RNA samples must be stored at low temperatures and handled with care to avoid introducing RNases, avoid shear stress and minimize hydrolysis. Even with such measures in place, a certain level of RNA degradation will take place once the RNA sample is thawed and put to use, and so the concentration of the RNA molecules of interest (i.e. intact, functional RNA molecules) will have decreased from the originally recorded concentration. It is therefore important to know the absolute and/or relative concentration of intact RNA molecules of interest in the sample when the sample is put to use.

In order to avoid an overestimation of the concentration of intact RNA molecules of interest in the sample, it is desirable to measure the level of RNA degradation that has taken place. In industry, the level of degradation of an RNA sample may be measured using capillary gel electrophoresis platforms. However, such platforms are not designed for analysing synthetic RNA and are lacking in sensitivity. For example, measurement of the integrity of RNA molecules based on size differences is especially challenging for large RNA molecules owing to the fact that hydrolysis of small RNA fragments from larger RNA molecules yields degradation products having sizes and/or charge-to-size ratios very close to that of the parent RNA. Hence, there exists a need for a more sensitive method for quantifying RNA degradation in an RNA sample.

The present inventors have developed a method of measuring RNA degradation which is highly sensitive. Specifically, the invention provides a method of analysing (or assessing) single-stranded RNA (ssRNA) in a sample, said method comprising:
(a) contacting the sample with a probe, wherein the probe binds to a target region of ssRNA in the sample;
(b) incubating the sample with a single-strand specific exoribonuclease; and
(c) detecting nucleotides and/or nucleosides generated by step (b), wherein the nucleotides and/or nucleosides comprise nucleotides and/or nucleosides from one or more RNA regions located outside the target region of ssRNA in the sample.

The method of the invention is based on the use of a single-strand specific exoribonuclease in combination with a probe, wherein the probe serves to block the degradation of the target site of the ssRNA by the exoribonuclease.

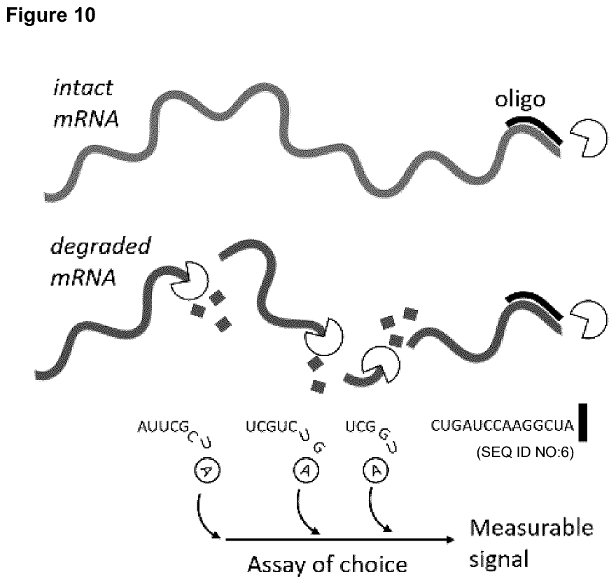

The degradation of ssRNA in a sample over time (whether enzymatically, chemically or otherwise) results in the formation of an ever-increasing number of 3′ and 5′ ends. The more 3′ and 5′ ends which are available for exoribonuclease to cleave, the more nucleotides and/or nucleosides are released by the action of the exoribonuclease on the sample. Thus, the level of nucleotides and/or nucleosides produced by incubating an ssRNA sample with exoribonuclease can be used to determine the level of degradation of the ssRNA in the sample (see FIG. 10).

However, it has been found that incubating the ssRNA with exoribonuclease alone is not sufficient to accurately determine the level of degradation. This is because a significant amount of nucleotides and/or nucleosides will be released from exoribonuclease incubation which is not related to the extent of degradation of the ssRNA in the sample. Specifically, these cleavage products originate from cleavage of the free ends present in the intact version of the ssRNA molecule in the sample (the 3′ end and/or the 5′ end, depending on the directionality of the exoribonuclease used in the method). Thus, when using exoribonuclease alone, there will always be a substantial level of background signal (or “noise”) generated.

By the addition of a probe which binds to the end of the ssRNA molecules in the sample (more specifically, a probe which binds to the 3′ end of the ssRNA molecules where a 3′→5′ exoribonuclease is used, or alternatively a probe which binds to the 5′ end of the ssRNA molecules where a 5′→3′ exoribonuclease is used), the exoribonuclease is prevented from cleaving regions of the ssRNA which are not indicative of degraded RNA, thus reducing the level of background signal and improving sensitivity. In other words, the single-strand specific exoribonuclease is unable to digest the free 3′ or 5′ end which is not the result of degradation because the probe forms a region of double stranded nucleic acid which is not a substrate for the single-strand specific exoribonuclease (or the probe blocks the exoribonuclease by shielding the bound region of ssRNA).

For example, in the case of measuring the level of degradation of mRNA in sample, the exoribonuclease can be a 3′→5′ exoribonuclease and the target region of the mRNA can be the poly(A) tail. The probe—for example a poly (dT) oligonucleotide, also referred to as an oligo (dT)—binds (or hybridises) to the poly(A) tail, thereby protecting it from degradation by the exoribonuclease.

The method of the invention can detect RNA degradation at low levels, potentially at a level of 0.1%, or even lower, in an RNA sample. The method of the present invention has greater sensitivity than methods of measuring RNA degradation which use capillary gel electrophoresis.

The method of the invention is also advantageous because the signal output generated from step (c) (e.g. light output) may be detected using either single throughput or high throughput detection hardware.

The method of the invention may also be applied to the measurement of 5′ capping efficiency and poly(A) tail length in mRNA molecules, as described further below.

The method of the present invention may also be used to measure the binding efficiency of a probe (which may be a candidate oligonucleotide probe or another candidate ssRNA-binding molecule or entity) to the target region. As described further below, this can be advantageous in the analysis of the degree of binding (or hybridisation or complementarity) of the probe to a target region within an ssRNA molecule of interest, and/or in the assessment of stem loop formation or hairpin formation (i.e. the propensity of the ssRNA of interest to form secondary structures such as hairpins). Thus, more specifically, the method of the present invention may be for assessing or measuring the degree of binding of the probe to the target region, or for assessing or measuring stem loop formation or hairpin formation in the ssRNA.

The method of the invention is a method of (or a method for) analysing ssRNA in a sample. Alternatively, the method of the invention can be viewed as a method of (or a method for) assessing, examining, inspecting, researching, reviewing or evaluating ssRNA in a sample. Preferably, the method of the invention is a method of quantifying or qualifying an RNA moiety of interest in an ssRNA sample.

The RNA moiety of interest may be intact RNA (e.g. mRNA) molecules or, and of course related thereto, the RNA moiety of interest may be RNA fragments generated as a result of RNA degradation. Thus, the moiety may be a whole RNA molecule or a fragment. Alternatively, the moiety may be the polyA tail where it is of interest to quantify the length. In a yet further embodiment, the RNA moiety may be a 5′ capping group and it may be of interest to know how effective a certain cap structure is at preventing digestion or to determine the proportion of mRNA molecules in a sample which incorporate a cap.

An “ssRNA sample” is a sample comprising ssRNA. The term “ssRNA” refers to an ssRNA molecule, or one or more ssRNA molecules, or a plurality of ssRNA molecules, as appropriate depending on the context in which it is used. Generally, the term “an ssRNA” means an ssRNA molecule or a type of ssRNA molecule, for example a type of ssRNA having a specific sequence or sharing a specific sequence or feature. For example, mRNA is a type of ssRNA molecule which may be analysed by the method of the invention. Different messenger RNA molecules may differ in certain regions, for example in the sequence of their protein coding regions; however, by virtue of being messenger RNA molecules, they all have certain features in common, for example they all encode a protein and generally all possess a poly(A) tail and a 5′ cap. Non-coding RNA (for example long non-coding RNA) may also be analysed by the method of the invention.

The term “ssRNA” as used herein also encompasses modified ssRNA. Modified ssRNA contains one or more modified nucleotides or regions. One or more or all of the nucleotides or regions in the molecule may be modified. Modified ssRNA may be modified in the sugar and/or nucleobase regions. Thus, the nucleotides and/or nucleosides produced by step (b) of the invention may be nucleotide analogues and/or nucleoside analogues.

Examples of sugar modifications include phosphorodiamidate Morpholino oligomer (PMO); 2′-O-methoxyethyl; 2′-O-methyl (2′-OMe); 2′-fluoro (2′-F); 2′-deoxy-2′-fluoroarabinonucleic acid (FANA); locked nucleic acid (LNA); unlocked nucleic acid (UNA); threose nucleic acid (TNA); 1,5-anhydrohexitol nucleic acid (HNA); cyclohexene nucleic acid (CeNA); and glycol nucleic acid (GNA). Examples of nucleobase modifications include 5-methoxyuridine; pseudouridine; N1-methylpseudouridine; 5-methylcytosine; abasic nucleosides; and 5-fluorobenzofuran-2′-deoxyuridine.

Modified mRNA is often used in mRNA-based therapeutics, and the ssRNA of the invention is preferably 5-methoxyuridine, pseudouridine or N1-methylpseudouridine modified mRNA.

The sample may be a product intended for medicinal, prognostic, diagnostic or research use which has been manufactured on a laboratory or industrial scale. The sample may be isolated from a human or other animal, plant or other organism. The sample may be purified or partially purified, but may not be.

The method of the invention may be at least partially automated, such that one or more steps of the method are performed without the involvement of a human (or do not require the involvement of a human). The method of the invention may be automated (or entirely automated or fully automated), for example such that all the steps of the method take place without the involvement of a human (or do not require the involvement of a human).

The method of the invention is an in vitro (and/or ex vivo) method.

The sample is preferably a solution comprising ssRNA and optionally buffer solution.

The method of the invention may be applied to the analysis of any ssRNA (i.e. any ssRNA molecule or type of ssRNA molecule, including modified RNA as discussed above). For example, the ssRNA may be synthetic or artificial ssRNA. Alternatively, the ssRNA may be non-synthetic, for example eukaryotic, bacterial, archaeal or viral ssRNA. Preferably the ssRNA is ssRNA comprising a poly(A) sequence at its 3′ end (i.e. a poly(A) tail) and/or a 5′ cap. More preferably, the ssRNA is mRNA, more preferably eukaryotic mRNA.

### Step (a)

The method of the invention comprises a step (a) of contacting the sample with a probe, wherein the probe binds to a target region of ssRNA in the sample.

The probe used in the method of the invention may be any molecule or entity which is capable of binding to ssRNA. The binding is typically an annealing or hybridising reaction but the probe may be or comprise, for example, an RNA binding protein. The RNA binding protein should specifically recognise and bind to the target region. For example, where the target region is the poly(A) tail, the RNA binding protein may be a poly(A) binding protein (PABP).

The step of contacting the sample with a probe may comprise combining (or mixing or admixing) the probe and the sample. Typically, the step of contacting the sample with a probe comprises adding a probe to the sample. Alternatively, the step of contacting the sample with a probe may comprise adding the sample to a solution comprising a probe.

In order for the probe to bind (or hybridise or anneal) to the target region of the ssRNA, heating and cooling steps may be employed. For example, the contacting step may comprise mixing the probe and ssRNA sample, heating the mixture, and then cooling the mixture. More preferably, this may involve heating at about 80° C. for two minutes, followed by cooling to room temperature. Such methods of facilitating binding (or hybridisation) are well known in the art, for example as used in multi-temperature polymerase chain reaction (PCR) protocols.

The probe may be an oligonucleotide, which term includes oligonucleotides comprising modified nucleic acid/nucleotides. Modified nucleic acid/nucleotides are structurally similar to naturally occurring RNA or DNA but contain one or more natural or synthetic linkages or modifications. The modification(s) may be located in any region of the compound, for example in the backbone region (more preferably in the sugar and/or phosphate region) and/or in the nucleobase region. Hence, the oligonucleotide may have alternative (or modified) backbone, nucleobase, or sugar-ring chemistry. Of course, in the context of the probe used in the present invention, any such modification must be such that the oligonucleotide (still) has the ability to bind (or sufficiently bind) the target region of ssRNA.

Examples of backbone modifications include phosphorothioate (PS); N3′→P5′ phosphoramidate (NP); 2′,5′-phosphodiester; and peptide nucleic acid (PNA). Examples of sugar modifications include phosphorodiamidate Morpholino oligomer (PMO); 2′-O-methoxyethyl; 2′-O-methyl (2′-OMe); 2′-fluoro (2′-F); 2′-deoxy-2′-fluoroarabinonucleic acid (FANA); locked nucleic acid (LNA); unlocked nucleic acid (UNA); threose nucleic acid (TNA); 1,5-anhydrohexitol nucleic acid (HNA); cyclohexene nucleic acid (CeNA); and glycol nucleic acid (GNA). Examples of nucleobase modifications include 5-methoxyuridine; pseudouridine; N1-methylpseudouridine; 5-methylcytosine; abasic nucleosides; and 5-fluorobenzofuran-2′-deoxyuridine. Such modifications may be advantageous because they may confer greater affinity to RNA, greater stability or greater resistance to degradation, and thus provide a probe with an improved ability to block the degradation of the target region of ssRNA by exoribonuclease.

Preferably the probe is a deoxyribonucleic acid (DNA) probe or RNA probe, more preferably a DNA probe. In other words, preferably the probe is a DNA molecule or an RNA molecule, more preferably a DNA molecule. Optionally the 5′ or 3′ phosphorylation status of the probe can be altered in order to render the probe unrecognizable to the exobribonuclease.

Preferably the probe is an oligonucleotide, i.e. a single-stranded polynucleotide containing a relatively small number of nucleotides. Thus, the oligonucleotide may have a length of up to 300, 290, 280, 270, 260, 250, 240, 230, 220, 210, 200, 190, 180, 170, 160, 150, 140, 130, 120, 110, 100, 90, 80, 70, 60, 50, 40, 30, 25, 20, 19, 18, 17, 16, or 15 nucleotides. Alternatively or in addition, the oligonucleotide may have a length of at least 15, 16, 17, 18, 19, 20, 21, 22, 23, 24, 25, 50, 100, 110, 120, 130, 140, 150, 160, 170, 180, 190, or 200 nucleotides. More preferably, the oligonucleotide has a length of 5 to 300, 5 to 250, 5 to 200, 5 to 150, 5 to 100, 5 to 50, or 5 to 25 nucleotides. Alternatively, the oligonucleotide may have a length of 10 to 300, 50 to 300, 100 to 300, 150 to 300, 200 to 300, or 250 to 300 nucleotides. Alternatively, the oligonucleotide may have a length of 5 to 250, 10 to 250, 50 to 250, 100 to 250, 150 to 250, or 200 to 250 nucleotides. Preferably, the oligonucleotide has a length of 15 to 100 nucleotides, more preferably 20 to 60 nucleotides, e.g. 25 to 45 nucleotides.

More preferably, the oligonucleotide is a DNA oligonucleotide or an RNA oligonucleotide, more preferably a DNA oligonucleotide.

The probe used in the method of the invention binds or hybridises to (or is suitable for or capable of binding or hybridising to) a target region of ssRNA in the sample.

Alternatively viewed, the probe used in the method of the invention is preferably complementary to a target region of ssRNA in the sample. The term “complementary” encompasses “partially complementary” as well as “fully complementary”. In preferred embodiments, the probe is fully complementary to the target region of ssRNA in the sample. As would be understood in the art, an oligonucleotide probe which is fully (i.e. 100%) complementary to the target region is an oligonucleotide which exhibits Watson-Crick base pairing with the target region across the whole sequence of the oligonucleotide probe.

In contrast, an oligonucleotide probe which is partially complementary to the target region may be defined as an oligonucleotide with at least 70, 75, 80, 85, 90, 95, 96, 97, 98, or 99% complementarity to the target region. Percentage complementarity means the percentage of bases (i.e. nucleobases or nitrogenous bases) in the probe which exhibit Watson-Crick base pairing with bases of the target region.

In embodiments, a saturating (or excess) quantity or concentration of probe may be used. This means at least a 1:1 molar (or stoichiometric) ratio of probe molecules to copies of the target region (i.e. a 1:1 molar ratio of probe molecules to copies of the intact ssRNA). Preferably an excess quantity or concentration of probe is used, more preferably a (or at least a) 2-fold, 3-fold, 4-fold, 5-fold, 10-fold, 50-fold, 100-fold, 500-fold, 1000-fold, 2000-fold, 3000-fold, 4000-fold, 5000-fold or 10,000-fold excess. Using a saturating or excess quantity or concentration of probe is advantageous because the greater the ratio of probe to copies of the target region, the greater percentage of copies of the target region will be bound or hybridised (or blocked) by the probe.

The “target region” will typically be a subsection of the ssRNA whose sequence has been pre-determined, to which the probe can bind. Typically, the sequence of the target region will be fully elucidated when the method of the invention is performed. However, it may only be necessary to elucidate the sequence of the target region to the extent that sufficient binding or hybridisation to the target region may be achieved, i.e. to the extent that the degradation of the target region (for example by exoribonuclease) is sufficiently prevented or blocked. For example, it may only be necessary for the probe to be partially complementary to the target region as discussed herein. Hence, in some embodiments the target region may be only partially elucidated when the method of the invention is performed.

The terms “target region of ssRNA” and “target region of the ssRNA” may be used interchangeably herein.

### Step (b)

The method of the invention comprises a step (b) of incubating the sample with a single-strand specific exoribonuclease.

Hence, where the term “exoribonuclease” is used herein to describe the exoribonuclease used in the invention, it is referring to a “single-strand specific exoribonuclease” as recited above.

The step of incubating the sample with an exoribonuclease may comprise combining (or mixing or admixing) the probe and the exoribonuclease. Typically, the step of incubating the sample with an exoribonuclease comprises adding an exoribonuclease to the sample. Alternatively, the step of incubating the sample with an exoribonuclease may comprise adding the sample to a solution comprising an exoribonuclease.

The incubation may be for at least (or up to) 5, 10, 15, 20, 25, 30, 35, 40, 45, 50, 55, 60, 65, 70, 75, 80, 85, 90, 95, 100, 110, 120, 130, 140, 150, 160, 170 or 180 minutes. Preferably, the incubation is for 5 to 180, 10 to 180, 15 to 180, 20 to 180, 25 to 180, 30 to 180, 35 to 180, 40 to 180, 45 to 180, 50 to 180, 60 to 180, 70 to 180, 80 to 180, 90 to 180, 100 to 180, 110 to 180, 120 to 180, 130 to 180, 140 to 180, 150 to 180, 160 to 180, or 170 to 180 minutes. Alternatively, the incubation may be for 5 to 180, 5 to 170, 5 to 160, 5 to 150, 5 to 140, 5 to 130, 5 to 120, 5 to 110, 5 to 100, 5 to 95, 5 to 90, 5 to 85, 5 to 80, 5, to 75, 5 to 70, 5 to 65, or 5 to 60 minutes. The incubation may conveniently be for 30 to 90 minutes.

The temperature or range of temperatures at which the incubation step should (or may most optimally) be performed will depend on the particular exoribonuclease used, because different exoribonucleases will have different temperature dependencies. It will be understood that it is within the remit of the skilled researcher to adjust the temperature of incubation step depending on the particular exoribonuclease that is used. For example, the incubation temperature can be 10 to 40° C. (e.g. 25 to 40° C.), preferably about 25° C. (for example when using exonuclease T as the exoribonuclease) or about 37° C. (for example when using PNPase or RNase R as the exoribonuclease).

Of course, “the sample” incubated with an exoribonuclease in step (b) is the sample which has been (or has previously been, or was, or was previously, or was earlier) contacted with a probe in step (a) as described herein. Thus, for the avoidance of doubt, it is noted that the steps of the method are performed in the order in which they are recited in claim 1 ((a), (b) then (c)).

An exoribonuclease is a protein which has exoribonuclease activity. An exoribonuclease degrades RNA by removing terminal nucleotides from either the 5′ end of RNA (i.e. a 5′→3′ exoribonuclease, or alternatively written as 5′-3′ exoribonuclease) or the 3′ end of RNA (i.e. a 3′→5′ exoribonuclease, or alternatively written as 3′-5′ exoribonuclease). In embodiments of the present invention, the exoribonuclease may be a 5′→3′ exoribonuclease or a 3′→5′ exoribonuclease, preferably a 3′→5′ exoribonuclease.

The exoribonuclease of the invention does not have (or has no significant) endonuclease (such as endoribonuclease) activity, i.e. the exoribonuclease of the invention is not (or cannot be classed as) an endonuclease. The exoribonuclease may have no measurable endonuclease activity or, in the case where it has some, albeit insignificant, endonuclease activity, it has at least 10 fold, 50 fold, 100 fold, 500 fold or 1000 fold greater exoribonuclease than endonuclease activity.

The exoribonuclease used in the invention is a single strand-specific exoribonuclease. As would be understood in the art, the term “single strand-specific exoribonuclease” means that the exoribonuclease of the invention specifically or preferentially cleaves (or degrades or digests) ssRNA over double stranded (ds) polynucleotides, for example dsRNA or DNA-RNA hybrid polynucleotides. The single-strand specific exoribonuclease may have no double strand exonuclease activity or, in the case where it has some double strand exonuclease activity, it may have at least 5 fold, 10 fold, 50 fold, 100 fold, 500 fold or 1000 fold greater single-strand exoribonuclease than double-strand (ds) exonuclease activity for any given concentration.

Specificity between ss and ds nucleic acid may be concentration dependent, but in the methods of the invention, the concentration of the reagents and enzymes (and reaction conditions) are selected to minimise ds exonuclease activity, such that in the method of the invention, the exoribonuclease exhibits at least 5 fold, 10 fold, 50 fold, 100 fold, 500 fold or 1000 fold greater single-strand exoribonuclease than double-strand (ds) exonuclease activity.

Thus, the catalytic activity (or the nucleic acid processing activity) of the single strand-specific exoribonuclease of the invention consists of (or consists primarily of) single strand-specific exo(ribo)nuclease activity. Alternatively viewed, in embodiments of the methods of the invention, the exoribonuclease does not demonstrate any (or any significant or strong) nucleic acid processing activity other than single strand-specific exoribonuclease activity.

The exoribonuclease may be a natural exoribonuclease or a synthetic exoribonuclease. The exoribonuclease may be a natural exoribonuclease or a synthetic exoribonuclease which has been modified (or mutated) to have increased specificity for ssRNA.

Exoribonucleases are classified into two subgroups by their mechanism of action, namely hydrolytic exoribonucleases and phosphorolytic exoribonucleases. Hydrolytic exoribonucleases cleave the nucleotide-nucleotide bond in RNA by hydrolysis (i.e. using water), thereby releasing nucleotide monophosphate (NMP). In contrast, phosphorolytic exoribonucleases cleave the nucleotide-nucleotide bond in RNA by phosphorolysis (i.e. using inorganic phosphate), thereby releasing nucleotide diphosphate (NDP). In some cases, a nucleoside (without phosphate) may be released as a result of exoribonuclease activity. For example, after spontaneous RNA hydrolysis (degradation), the 5′ fragment typically contains a 2′,3′-cyclic-phosphate. Consequently, the 3′ fragment does not have a phosphate. When an exoribonuclease acts upon this last fragment, one nucleoside is released.

Thus, in embodiments of the method of the invention, the exoribonuclease is a hydrolytic exoribonuclease or a phosphorolytic exoribonuclease. Preferably, the hydrolytic exoribonuclease is exonuclease T or RNAse R. Preferably, the phosphorolytic exoribonuclease is polynucleotide phosphorylase (PNPase). In embodiments, the exoribonuclease is a nucleotide-producing exoribonuclease, or is capable of producing nucleotide(s) from RNA cleavage activity.

Using PNPase is advantageous when used in conjunction with the Lucipac A3 assay (as described elsewhere herein) because the Lucipac A3 assay generates inorganic phosphate (Pi) which in turn stimulates PNPase to increase its degradation activity. However, eventually an equilibrium is reached, where a high quantity of NDPs have been generated (on average, 25% will be ADP) which inhibit the PNPase-mediated degradation. This feedback dampens the effect of PNPase so that the system does not become saturated.

Using PNPase is advantageous in other circumstances due to its ability to recognise 2′,3′-cyclic phosphate. Under certain circumstances of RNA degradation, 2′,3′-cyclic phosphate is formed, i.e. the 3′ end formed from the cleavage of the RNA is a 2′,3′-cyclic phosphate end.

When PNPase acts upon a 2′,3′-cyclic phosphate end, nucleoside diphosphate is liberated as normal (i.e. like when PNPase acts upon a hydroxyl group 3′ end or a phosphate group 3′ end), which can then be detected by one of several known methods, for example any one of the detection methods described elsewhere herein. Thus, in embodiments of the method of the invention where the exoribonuclease is a 3′→5′ exoribonuclease, it is particularly advantageous for the exoribonuclease to be an exoribonuclease which recognises (or is able to act upon, or cleave or digest or process) 2′,3′-cyclic phosphate (alternatively referred to as a 2′,3′-cyclic phosphate, or a 2′,3′-cyclic phosphate end, or a 2′,3′-cyclic phosphate 3′ end, or a 2′,3′-cyclic phosphate at the 3′ end of ssRNA).

PNPase is available from Sigma.

However, it should also be noted that, when conducting the method of the invention on an ssRNA sample which comprises (or may comprise, or is suspected of comprising) one or more ssRNA molecules which comprise a 2′,3′-cyclic phosphate end, it is not necessary to limit the particular type of exoribonuclease that is used. Rather, the 2′,3′-cyclic phosphate end can be addressed by “opening up” the 2′,3′-cyclic phosphate end, i.e. converting the 2′,3′-cyclic phosphate 3′ end into a 3′ end recognisable to all (or most) exoribonucleases, such as a hydroxyl group 3′ end or a phosphate group 3′ end as described above. This conversion may be achieved in a number of ways. In water, 2′,3′-cyclic phosphate exists in an equilibrium state with 2′-phosphate and 3′-phosphate, and this equilibrium is dependent upon the conditions of the reaction. Opening up (or converting) of the 2′,3′-cyclic phosphate end may be achieved chemically, for example by incubating the ssRNA sample with HCl. Alternatively or in addition, opening up (or converting) of the 2′,3′-cyclic phosphate end may be achieved enzymatically, for example using T4 polynucleotide kinase-phosphatase in the absence of ATP (Das and Shuman, “Mechanism of RNA 2′,3′-cyclic phosphate end healing by T4 polynucleotide kinase-phosphatase”, Nucleic Acids Research, 2013, Vol. 41, No. 1, 355-365).

Thus, the method of the invention can be applied to an ssRNA sample which comprises (or may comprise, or is suspected of comprising) one or more ssRNA molecules which comprise a 2′,3′-cyclic phosphate end. By “one or more ssRNA molecules which comprise (or may comprise) a 2′,3′-cyclic phosphate end”, it is meant one or more ssRNA molecules wherein, in the 3′ terminal nucleotide (or modified nucleotide) of the ssRNA molecule, the 2′ and 3′ positions of ribose are bridged by phosphate.

In embodiments where the exoribonuclease is a hydrolytic exoribonuclease, the detecting step (i.e. step (c)) comprises detecting nucleoside monophosphate. Thus, in embodiments, the method of the invention comprises the steps of: (a) contacting the sample with a probe, wherein the probe binds (e.g. hybridises) to a target region of ssRNA in the sample; (b) incubating the sample with a hydrolytic single strand specific exoribonuclease; and (c) detecting nucleoside monophosphate generated by step (b), wherein the nucleoside monophosphate comprises nucleoside monophosphate from one or more RNA regions located outside the target region of ssRNA in the sample.

In embodiments where the exoribonuclease is a phosphorolytic exoribonuclease, the detecting step (i.e. step (c)) comprises detecting nucleoside diphosphate. Thus, in embodiments, the method of the invention comprises the steps of: (a) contacting the sample with a probe, wherein the probe binds (e.g. hybridises) to a target region of ssRNA in the sample; (b) incubating the sample with a phosphorolytic single strand specific exoribonuclease; and (c) detecting nucleoside diphosphate generated by step (b), wherein the nucleoside diphosphate comprises nucleoside diphosphate from one or more RNA regions located outside the target region of ssRNA in the sample.

Optionally, both a hydrolytic exoribonuclease and a phosphorolytic exoribonuclease may be used in the method of the invention, in which case the detecting step comprises detecting both nucleoside monophosphate and nucleoside diphosphate.

In some embodiments, a saturating (or excess) concentration of exoribonuclease may be used. This means a quantity or concentration of exoribonuclease at which cleavage of the ssRNA occurs at the maximum rate. This would generally be understood to be a quantity or concentration of exoribonuclease which equals or exceeds the number of substrates in the ssRNA sample (a substrate being a 5′ end of ssRNA in the case of a 5′→3′ exoribonuclease, or a 3′ end of ssRNA in the case of a 3′→5′ exoribonuclease). The number of substrates may be estimated based on knowledge of the RNA sample. Using a saturating quantity or concentration of exoribonuclease is advantageous because it enables rapid production of nucleotides and/or nucleosides (depending on the exoribonuclease(s) used) and thus rapid determination of the level of ssRNA degradation. Saturating the RNA sample with exoribonuclease also allows quantifying the number of free RNA ends by measuring the rate of increase in concentration of nucleotides and/or nucleosides over time.

### Step (c)

The method of the invention comprises a step (c) of detecting (or measuring or observing or monitoring) nucleotides and/or nucleosides generated by step (b), wherein the nucleotides and/or nucleosides comprise nucleotides and/or nucleosides from one or more RNA regions located outside the target region of ssRNA in the sample.

Optionally, the detecting step uses an enzyme-reagent mixture (i.e. step (c) comprises detecting (or measuring or observing or monitoring) nucleotides and/or nucleosides generated by step (b) using an enzyme-reagent mixture).

The nucleotides and/or nucleosides detected during the detecting step may be natural (or standard) nucleotides and/or nucleosides. For example, the nucleotides and/or nucleosides detected during the detecting step may be adenosine, uridine, cytidine or guanosine, or a nucleoside monophosphate of one of those nucleosides, or a nucleoside diphosphate of one of those nucleosides, or combinations thereof (i.e. adenosine, adenosine monophosphate (AMP), adenosine diphosphate (ADP), uridine, uridine monophosphate (UMP), uridine diphosphate (UDP), cytidine, cytidine monophosphate (CMP), cytidine diphosphate (CDP), guanosine, guanosine monophosphate (GMP), guanosine diphosphate (GDP), or combinations thereof).

Alternatively or in addition, the nucleotides and/or nucleosides detected during the detecting step may be modified nucleotides and/or nucleosides. For example, the nucleotides and/or nucleosides detected during the detecting step may be 5-methoxyuridine; pseudouridine; N1-methylpseudouridine; 5-methylcytosine; abasic nucleosides; or 5-fluorobenzofuran-2′-deoxyuridine, or a nucleoside monophosphate of one of those nucleosides, or a nucleoside diphosphate of one of those nucleosides, or combinations thereof. Alternatively, the nucleotide and/or nucleoside may be (or may be derived from the degradation of) any of the other modified nucleic acids described elsewhere herein or combinations thereof.

Preferably, the nucleotides and/or nucleosides detected during the detecting step are nucleosides, nucleoside monophosphates, nucleoside diphosphates, or combinations thereof; more preferably adenosine, adenosine monophosphate, adenosine diphosphate, or combinations thereof.

Preferably, the nucleotides and/or nucleosides detected during the detecting step are nucleoside monophosphates, nucleoside diphosphates, and combinations thereof; preferably adenosine monophosphate and/or adenosine diphosphate.

As is evident from the above discussion, the nucleotides and/or nucleosides detected during the detecting step may be a single type (or species) of molecule or a mixture of multiple types (or species) or molecule. In order to detect multiple types (or species) of molecule during the detecting step, it may be appropriate to use multiple detection methods simultaneously or sequentially. Thus, just one base (whether in nucleoside or nucleotide form) may be detected, and that is preferred, but detection may be of more than one type of base, even of all 4 bases (whether in nucleoside or nucleotide form).

Of course, the nucleotides and/or nucleosides detected during the detecting step are individual nucleotides and/or nucleosides, as is the normal meaning of these terms, in contrast for example to polynucleotides.

The output signal generated in step (c) is proportional to the amount (or level) of nucleotides and/or nucleosides generated by step (b).

The nucleotides and/or nucleosides may be detected by any suitable detection method known in the art.

Mass spectrometry and nuclear magnetic resonance (NMR) are tools which may be applied to the detection and quantification of any nucleotide and/or nucleoside. However, it may be preferable to use an enzyme-based method of detection or an immunoassay, and many kits and reagents for such methods are available in the art.

For example, the nucleotides and/or nucleosides may be detected using a luminescence or fluorescence based method. In the case of AMP and/or ADP, these molecules may be detected using a Lucipac A3 enzyme mix as described for example in Bakke and Suzuki 2018 (Journal of Food Protection, Vol. 81, No. 5, 2018, Pages 729-737) and Bakke et al. 2020 (Journal of AOAC INTERNATIONAL, 103 (4), 2020, 1090-1104). In the Lucipac A3 protocol, AMP is converted to ATP in a reaction catalysed by pyruvate orthophosphate dikinase (PPDK), and ADP is converted to ATP in a reaction catalysed by pyruvate kinase. The ATP generated through these two reactions is used in a reaction catalysed by luciferase to generate light.

The luciferase-catalysed reaction converts ATP (i.e. the ATP generated by the PPDK- and PK-catalysed reactions) to AMP. The AMP produced from the luciferase-catalysed reaction is then converted (or “recycled”) back to ATP by the PPDK-catalysed reaction. The resultant ATP is then used again in the luciferase-catalysed reaction, leading to the generation of more light and AMP. The resultant AMP is then recycled into ATP again, and the cycle repeats.

This process of AMP recycling leads to the generation of a constant light intensity which is proportional to the amount of AMP and ADP in the sample. The AMP and ADP in the sample can thus be quantified by measuring the light intensity using a luminometer, for example by providing an output of Relative Light Units (RLU) or by measuring the number of released photons per second (p/s).

Guanosine diphosphate (GDP) may be also detected by a luminescence-based method, for example Promega GDP-Glo assay (VA1090). Similarly, uridine diphosphate (UDP) may be detected by Promega UDP-Glo assay (V6961).

Adenosine may be detected by a fluorometric method, for example using the CELLTECHGEN adenosine assay kit, or the BIOVISION adenosine assay kit (K327-100).

Thus, in embodiments, the detecting step provides a light output. In preferred embodiments, the detecting step uses an enzyme-reagent mixture, preferably the mixture comprising luciferase, more preferably also pyruvate orthophosphate dikinase and/or pyruvate kinase.

Alternatively, AMP and/or ADP may be detected using an NAD+/NADH coupled assay, or an NADP+/NADPH coupled assay. For example, a traditional method of detecting ADP is to use the ADP to convert phosphoenol pyruvate (PEP) to pyruvate in a reaction catalysed by pyruvate kinase, and then to convert the pyruvate to lactate with simultaneous oxidation of NADH to NAD+. NADH absorbs light at 340 nm, while NAD+ does not. Thus, the change in concentration of NADH can be detected by measuring the light absorbance of the sample at 340 nm. Alternative methods of measuring the concentration of NAD+ or NADH are also known in the art.

Similarly, GDP may be detected by several other methods including the Profoldin micromolar GDP assay kit (MGD100K-PF), which can also be used to detect ADP.

Alternatively, nucleotides and/or nucleosides may be detected using an immunodetection method.

For example, AMP and/or ADP may be detected using anti-AMP and/or anti-ADP antibodies, for example using the Transcreener AMP² assay or the Transcreener ADP² assay (described for example in Kleman-Leyer et al. Characterization and optimization of a red-shifted fluorescence polarization ADP detection assay. Assay Drug Dev Technol 2009; 7 (1):56-67). The Transcreener assays uses an AMP or ADP analog which is covalently fused to a fluorescent tracer. The nucleotide analog-tracer fusion molecule is bound to an anti-AMP or anti-ADP antibody. If there is enzymatic activity resulting in the formation of ADP, then the fusion molecule is displaced by the ADP. Displacement of the tracer from the anti-ADP or anti-AMP antibody causes a change in the fluorescence properties of the tracer which can then be detected using standard apparatus.

Similarly, GDP may be detected using the Transcreener GDP FP assay (Bellbrook labs 3009-1k).

Alternatively, nucleotides and/or nucleosides may be detected using an enzyme-coupled detection method.

For example, AMP and/or ADP may be detected using an enzyme-coupled detection method, for example using the ADP-Quest assay (described for example in Charter et al. A generic, homogenous method for measuring kinase and inhibitor activity via adenosine 5′-diphosphate accumulation. J Biomol Screen 2006; 11:390-399). In the ADP-Quest assay, ADP is used to drive a cascade of detection enzymes that ultimately produces a fluorescence signal. More specifically, ADP is converted to ATP in a reaction catalysed by pyruvate kinase, with the concomitant conversion of phosphoenolpyruvate to pyruvate. The pyruvate is then converted to hydrogen peroxide by pyruvate oxidase. The hydrogen peroxide is then reacted with Amplex Red in a reaction catalysed by peroxidase to produce the fluorescent compound resorufin. The concentration of resorufin is then determined by fluorescence, using standard apparatus.

In some embodiments, and as mentioned previously herein, the invention may be used for measuring or quantifying ssRNA degradation in a sample. This can be achieved using either a 5′→3′ exoribonuclease or a 3′→5′ exoribonuclease, or both.

In order to measure ssRNA degradation by the method of the invention using a 3′→5′ exoribonuclease, a probe should be used which binds (or hybridises) to a sequence at or near the 3′ end of the ssRNA, i.e. the “target region” of ssRNA is (or is a sequence) at or near the 3′ end of the ssRNA. Thus, in embodiments, the exoribonuclease is a 3′→5′ exoribonuclease, and the target region of ssRNA is at or near the 3′ end.

Thus, if the ssRNA for which degradation is to be measured is an ssRNA comprising a poly(A) tail, for example mRNA, then degradation can be measured by the method of the invention by using a 3′→5′ exoribonuclease, together with a probe which binds (or hybridises) to the poly(A) tail or a portion thereof. Thus, in embodiments, the exoribonuclease is a 3′→5′ exoribonuclease, the ssRNA comprises a poly(A) tail, and the target region of ssRNA is said poly(A) tail. Hence, the probe is one which binds or hybridises to (or is capable of binding or hybridising to) said poly(A) tail. For example, the probe may be an oligonucleotide DNA probe comprising or consisting of a poly (T) sequence, preferably consisting of a poly (T) sequence. Alternatively, the probe may be an oligonucleotide RNA probe comprising or consisting of a poly (U) sequence, preferably consisting of a poly (U) sequence. In this way, the incubation of the probe with the ssRNA leads to binding of the probe to the poly(A) tail to form a duplex, thereby protecting the poly(A) tail from degradation by the single-strand specific exoribonuclease of the invention.

In order to measure ssRNA degradation by the method of the invention using a 5′→3′ exoribonuclease, a probe should be used which binds (or hybridises) to a sequence at or near the 5′ end of the ssRNA, i.e. the “target region” of the ssRNA is (or is a sequence) at or near the 5′ end of the ssRNA. 5′→3′ exoribonuclease activity is generally prevented by the presence of a 5′ cap; however not all mRNA in the sample may possess a 5′ cap. Thus, a probe should be used which protects the 5′ ends of mRNA molecules which do not possess a 5′ cap (i.e. uncapped mRNAs), such that degradation of such molecules at the 5′ end by 5′→3′ exoribonuclease is prevented. Thus, in embodiments, the exoribonuclease is a 5′→3′ exoribonuclease, and the target region of ssRNA is at or near the 5′ end of the ssRNA. Optionally, an additional probe can be used whose target region of ssRNA is immediately upstream of the poly(A) tail in order to prevent excessive signal generation.

The term “upstream of” takes its normal meaning in the art, i.e. “5′ of” or “5′ with respect to”. This term contrasts with “downstream of”, which also takes its normal meaning in the art, i.e. “3′ of” or “3′ with respect to”.

As used herein, where it is contemplated that the target region of ssRNA is “immediately upstream” of a polynucleotide region in question, e.g. of the poly(A) tail, it is considered that the target region of ssRNA comprises or consists of nucleotides which are within 10, 9, 8, 7, 6, 5, 4, 3, 2, 1, or 0 nucleotides of the polynucleotide region in question, preferably 0 nucleotides (i.e. the first nucleotide upstream of the polynucleotide region in question, e.g. of the poly(A) tail).

As used herein, where it is contemplated that the target region is “near” a polynucleotide region in question, e.g. the poly(A) tail or 5′ end, it is considered that the target region of ssRNA is within 50, 40, 30, 20 or 10 nucleotides of the polynucleotide region in question, preferably within 10 or 5 nucleotides.

In some embodiments, the method of the invention may be used for measuring the binding efficiency (or binding affinity or hybridisation efficiency or hybridisation affinity) of the probe to the ssRNA (the target region of ssRNA).

Binding efficiency of the probe to the target region of ssRNA may be suboptimal due to a lack of full complementarity between an oligonucleotide probe and the target region. Thus, more specifically, in some embodiments the invention may be used to determine the extent of complementarity between the probe and the ssRNA (or the target region of ssRNA), for example where the full sequence of the probe (where the probe is an oligonucleotide) and/or of the ssRNA (or the target region of ssRNA) has not been fully elucidated (i.e. the sequence of the probe and/or of the ssRNA (or the target region of ssRNA) have only been partially elucidated).

Alternatively or in addition, binding efficiency of the probe to the target region of ssRNA may be suboptimal due to steric hindrance of binding of the probe to the target region, for example due to secondary structure formation (e.g. formation of a hairpin or hairpins) in the ssRNA in, and/or around (e.g. near, or immediately upstream of, or immediately downstream of), the target region of ssRNA. Thus, more specifically, in some embodiments the invention may be used to determine the extent of secondary structure formation in and/or around the target region of ssRNA, preferably wherein the secondary structure formation is hairpin (or stem loop) formation. In such embodiments, it is preferred to use an exoribonuclease which is not inhibited by stem loops, for example RNase R.

Probes which are not oligonucleotides or analogues thereof may also have variable binding affinities for a target region within a ssRNA molecule and the methods of the invention may be deployed to investigate binding affinity of a candidate probe molecule and/or to screen putative probe molecules to identify those with strong(er) affinity.

A 5′ cap is a non-standard nucleotide found at the 5′ end of precursor mRNA and other primary RNA transcripts. In eukaryotes it consists of a methylated guanine nucleotide attached to the rest of the mRNA by an atypical 5′-5′ triphosphate linkage.

The invention may be used for measuring or quantifying mRNA capping efficiency in a sample (i.e. in an mRNA sample) i.e. the proportion of mRNA molecules in a sample incorporating a 5′ cap. In such embodiments, a 5′→3′ exoribonuclease is used. The 5′→3′ exoribonuclease will digest the 5′ end of uncapped mRNA. However, the 5′→3′ exoribonuclease will be unable to digest the 5′ end of capped mRNA, since such 5′ ends are protected from degradation by the 5′ cap. Preferably, the 5′→3′ exoribonuclease is Xrn-1.

Capping efficiencies in mRNA are typically >95%, and hence require an efficient assay to detect small differences in capping efficiency. Thus, a probe is required downstream (though not immediately downstream) of the 5′ end of the mRNA in order to prevent excessive signal generation. Thus, in embodiments of the method of the invention, the exoribonuclease is a 5′→3′ exoribonuclease, and the target region of ssRNA is (or is located) downstream but not immediately downstream of the 5′ end of the mRNA. Preferably, the target region of ssRNA is (or is located) at least 5, 6, 7, 8, 9, 10, 20, 30, 40, 50, 60, 70, 80, 90 or 100 nucleotides downstream of the 5′ end of the mRNA. Preferably the target region is located (starts) no more than 50, 60, 70, 100, 150, 200 or 250 nucleotides downstream of the 5′ end of the mRNA.

In some embodiments, the invention can be used for measuring or quantifying poly(A) tail length. In such embodiments, a 3′→5′ exoribonuclease is used in order to digest the poly(A) tail. However, in the absence of a blocking probe, the exoribonuclease will additionally digest mRNA nucleotides upstream of the poly(A) tail and this is undesirable. In order to prevent or minimise this undesirable digestion, a probe may be used which binds immediately upstream of the poly(A) tail (i.e. a probe for which the target region of ssRNA is immediately upstream of the poly(A) tail). Thus, in embodiments, the exoribonuclease is a 3′→5′ exoribonuclease, the ssRNA comprises a poly(A) tail, and the target region of ssRNA is immediately upstream of said poly(A) tail.

It will be understood that if the probe only binds near the final nucleotide before the start of the poly(A) tail, that the readout can be distorted by adenine nucleotides present in the RNA between the end of the probe and the start of the poly(A) tail. Such residues could contribute to the signal generated by the enzyme-reagent mixture. “Immediately” will be understood in that context, as preferably requiring no nucleotides between the end of the probe and the start of the poly(A) tail, but if there are a few nucleotides in that zone, especially if none of them are adenine, then the analysis will still be useful.

Alternatively, part of the probe may be complementary to the RNA sequence directly upstream of the poly(A) tail and the other part of the probe may be complementary to a region of the poly(A) tail. Thus, alternatively worded, the probe binds (or hybridises) around the 5′ end of the poly(A) tail. Hence, in embodiments, the exoribonuclease is a 3′→5′ exoribonuclease, and the target region of ssRNA is a region spanning nucleotides at the 5′ end of the poly(A) tail and nucleotides immediately upstream of the poly(A) tail.

The method of the invention may comprise performing a further method as described above (or further method steps) on the same sample material, in which step (a) of the method of the invention is not performed but steps (b) and (c) of the method of the invention are performed. A comparison of the results obtained in these two methods may be performed, in which the second method (without step (a)) operates as a control step.

Thus, optionally, the method of the invention also comprises comparing the level of nucleotides and/or nucleosides detected in step (c) of a method in which step (a) was included, with the level of nucleotides and/or nucleosides detected in step (c) of a method in which step (a) was not included. The comparison may involve providing (or calculating or determining) a ratio of the two levels. Preferably the same nucleotide or nucleoside is detected in the control method as is in the method of the invention.

The further method (steps) serve as a control or baseline by which the detected levels (for example luminescence levels, for example where the Lucipac A3 protocol is used) of nucleotides and/or nucleosides generated by step (b) from the original method steps can be normalised. The use of the further method steps, while not essential, can be advantageous, for example enabling a more meaningful output irrespective of the particular apparatus or parameters which are used.

For example, in an embodiment of the method of the invention for measuring or quantifying the efficiency of binding (or hybridisation) of a probe to target ssRNA, the further method steps involve incubating the sample with (i) and without (ii) oligonucleotides. The fraction of (i)/(ii) will then inform the user on binding efficiency of the probe to the RNA. Specifically, no or weak binding will result in a ratio of (or close to) 1, whereas strong binding will result in ratio close (or closer) to 0.

In embodiments, it may be desirable to take into account background signal in calculations. Background signal (or “noise”) may be generated for example by trace contaminant nucleotides. This signal can be quantified and thus taken into account by measuring the signal (e.g. luminescence signal) generated by the same protocol in the absence of the mRNA sample. Thus, in the formula (i)/ii) described above, the values of (i) and (ii) may exclude background signal.

The percentage degradation can be calculated using the following formula, this time with background signal taken into account explicitly in the equation:

Percentage degradation=(degradation−background)/(no probe−background)

where “degradation” is the signal (for example a luminescence signal) from the degraded sample following incubation with exoribonuclease and subsequent detection (for example using the Lucipac A3 assay), “background” is the signal generated in the absence of the mRNA sample (the signal may be due for example to trace contaminant nucleotides), and “no probe” is the signal determined from the degraded sample in the absence of probe.

Instead of determining and using the signal produced for the ssRNA sample in the absence of a probe as the comparator, one may alternatively determine and use the signal produced for a version of the ssRNA sample which is (or is assumed) to be 100% intact (i.e. have 0% degradation) in the presence of the probe (i.e. performing all of steps (a) to (c) of the invention as normal but with an intact sample). An example of such a sample may be a freshly thawed or newly generated version or aliquot of the ssRNA sample in question, or a version or aliquot of the ssRNA sample which has otherwise not been exposed to degrading conditions.

In another aspect, the invention provides a kit for performing a method of ssRNA analysis as defined herein, said kit comprising the probe of the invention and the exoribonuclease of the invention as defined herein.

Optionally, the kit comprises a buffer solution. The buffer solution may be a buffer solution for (or suitable for) the probe and/or the exoribonuclease. Optionally, the kit comprises means for detecting nucleotides and/or nucleosides, for example an enzyme-reagent mixture as defined herein.

Preferred embodiments and additional features of the method of the invention apply, mutatis mutandis, to this aspect of the invention.

Throughout the present specification, where the terms “comprising”, “comprise”, “comprises”, “has” or “having” or other equivalent terms are used herein, the terms “consisting of”, “consist of” and “consists of” are also alternatively contemplated. Methods comprising certain steps also include, where appropriate, methods consisting of these steps.

Throughout the present specification, the terms “a” and “an” are used in the sense that they mean “at least one”, “at least a first”, “one or more” or “a plurality” of the referenced components or steps, except in instances wherein an upper limit is thereafter specifically stated.

The invention will now be further described in the following non-limiting Examples with reference to the following figures.

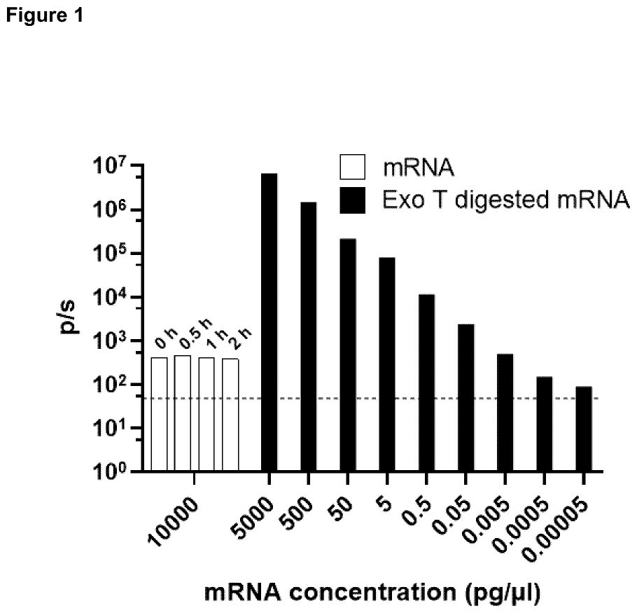

FIG. 1, showing results of Example 1, is a graph displaying the signal output from a Lucipac A3 assay conducted on mRNA samples of varying mRNA concentration, with or without prior incubation of the sample with exonuclease T.

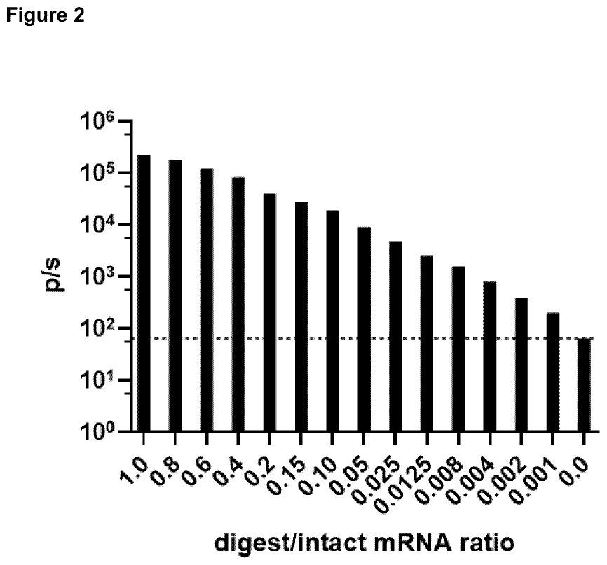

FIG. 2, showing results of Example 2, is a graph displaying the signal output from a Lucipac A3 assay conducted on mRNA sample of varying ratios of digested (degraded) mRNA to intact mRNA.

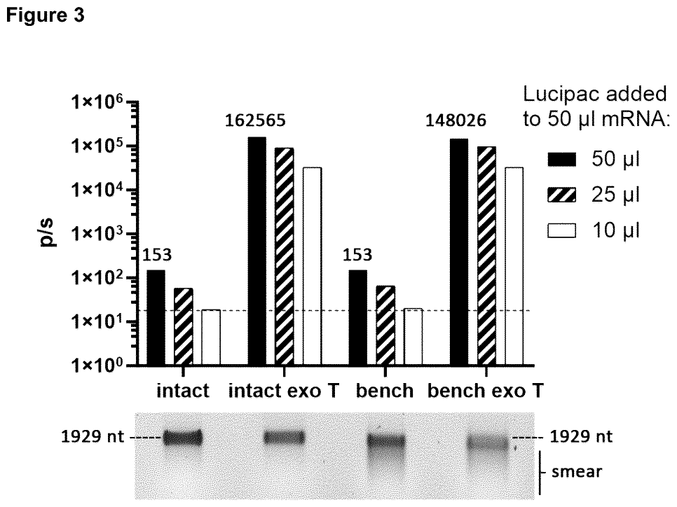

FIG. 3 shows results of Example 3. The upper part of the figure contains a graph displaying the signal output from a Lucipac A3 assay conducted on: a mRNA sample freshly thawed (“intact”); a mRNA sample freshly thawed and subsequently incubated with exonuclease T (“intact exo T”); an mRNA sample incubated at room temperature for 1 week (“bench”); and an mRNA sample incubated at room temperature for 1 week and subsequently incubated with exonuclease T (“bench exo T”). The lower part of the figure contains an agarose gel of each sample, wherein smear is indicative of mRNA degradation.

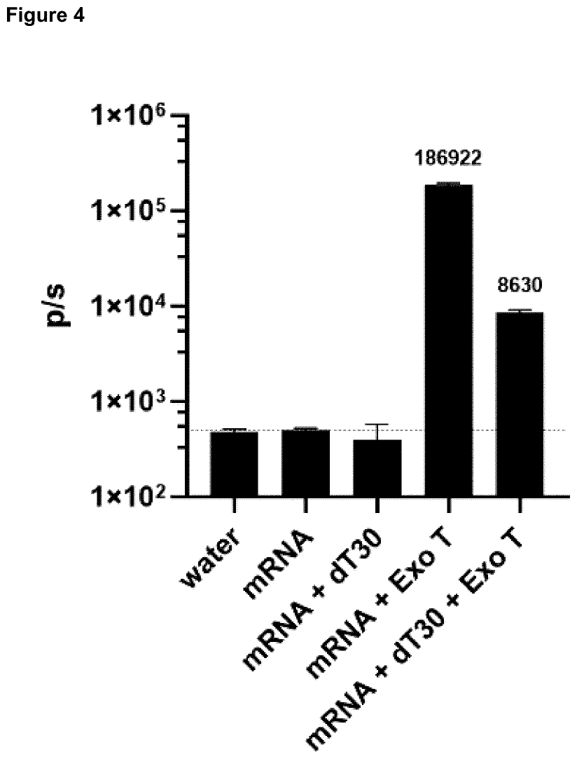

FIG. 4, showing results of Example 4, is a graph displaying the signal output from a Lucipac A3 assay conducted on: water; mRNA; mRNA incubated with dT30 (ssDNA consisting of a sequence of 30 thymine residues); mRNA incubated with dT30; mRNA incubated with exonuclease T; and mRNA incubated with dT30 and exonuclease T.

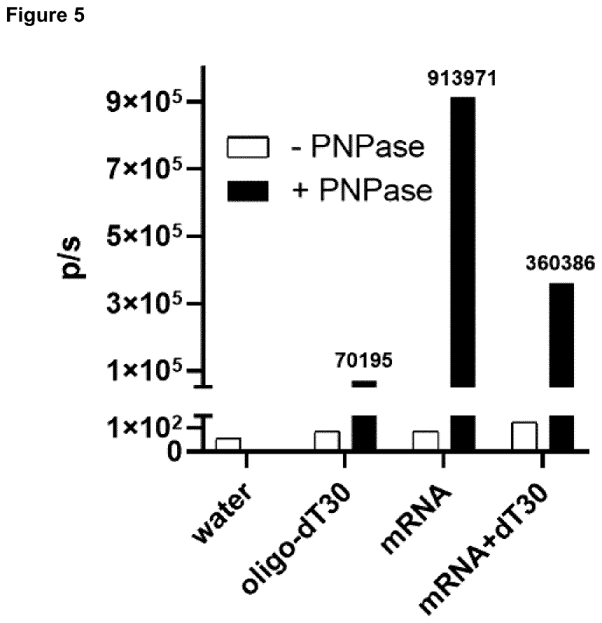

FIG. 5, showing results of Example 5, is a graph displaying the signal output from a Lucipac A3 assay conducted on: water; dT30; mRNA; and mRNA incubated with dT30; all with or without incubation with PNPase.

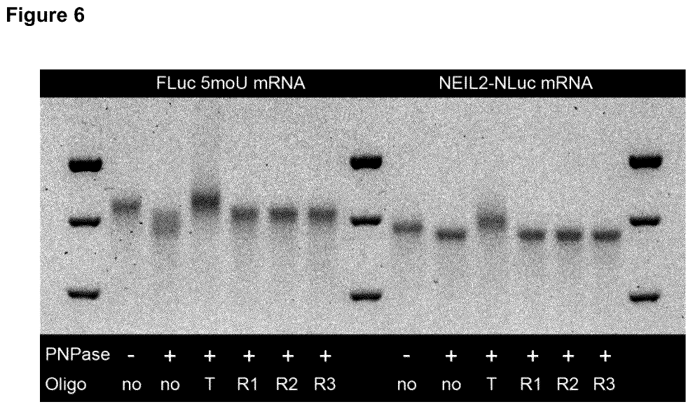

FIG. 6, showing results of Example 6, is an image of an agarose TAE gel displaying the molecular weight (and thus the level of degradation) of different samples of mRNA in the presence or absence of dT30 (T) or random sequence oligodeoxynucleotode (R1, R2, R3), and with or without subsequence incubation with PNPase.

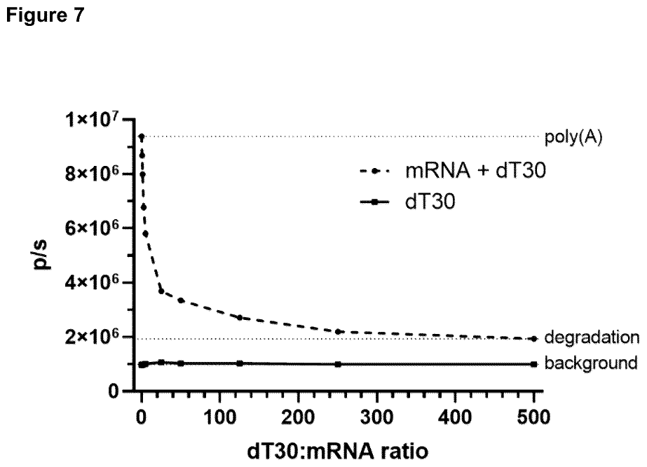

FIG. 7, showing results of Example 7, is graph displaying the output signal from a Lucipac A3 assay as the concentration ratio of dT30 to mRNA is increased.

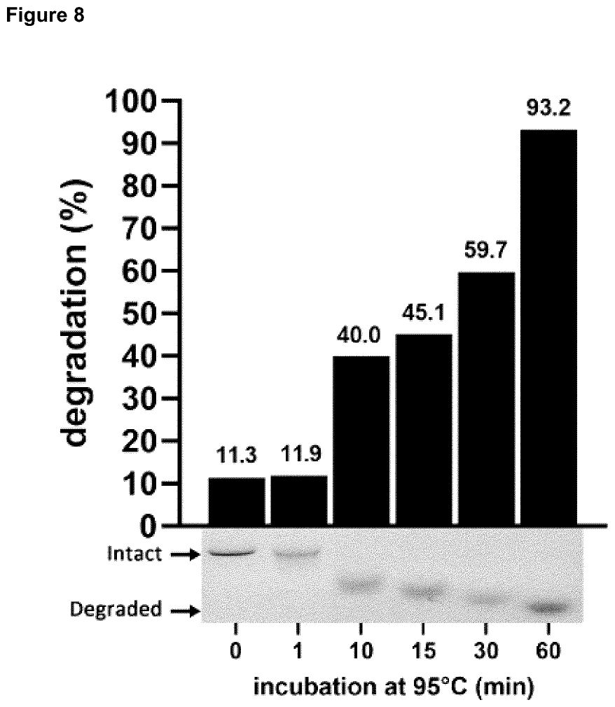

FIG. 8 shows results of Example 8. The upper part of the figure is a graph displaying the calculated percentage degradation derived from a PNPase-based assay using the formula: Percentage degradation=(degradation-background)/(poly(A)-background). The same mRNA was used as input for the degradation assay and as input for the gel assay. The lower part of the figure is an image of a TAE agarose gel displaying the molecular weight (and thus level of degradation) of an originally intact mRNA sample after 0, 1, 10, 15, 30 or 60 minutes of incubation at 95° C.

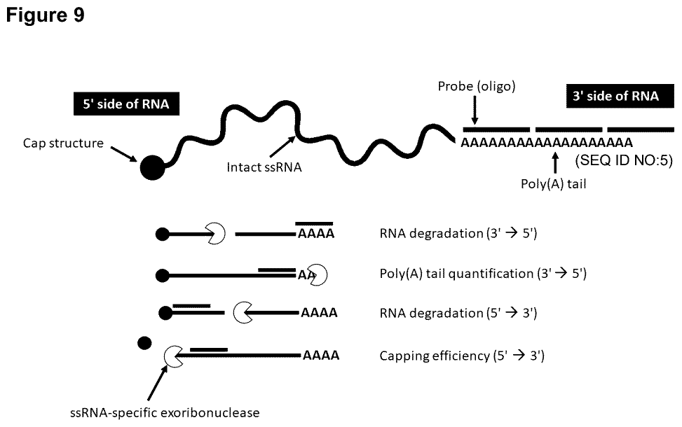

FIG. 9 provides a brief schematic of four embodiments of the invention as described above, namely measuring RNA degradation using 3′→5′ exoribonuclease, measuring RNA degradation using 5′→3′ exoribonuclease, measuring capping efficiency, and measuring poly(A) tail length. It will of course be understood that these schematic diagrams are provided for illustrative purposes and are non-limiting.

FIG. 10 provides a more detailed schematic of one embodiment of FIG. 9, namely measuring RNA degradation using 3′→5′ exoribonuclease. It will of course be understood that this schematic diagram is provided for illustrative purposes and is non-limiting.

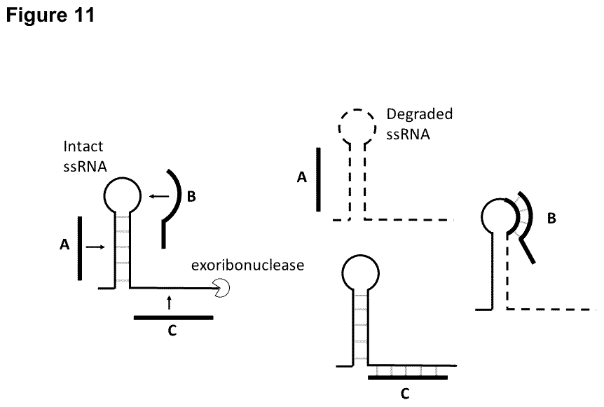

FIG. 11 provides a schematic of how the invention can be used to determine secondary structure of ssRNA and can also be used to assess the binding affinity of a probe to a ssRNA. A, B and C are oligonucleotide probes that bind ssRNA that contains secondary structure (a stem-loop). Probe A does not bind very well and so addition of an exoribonuclease will completely degrade the ssRNA. Probe B binds moderately well and so the ssRNA will be degraded partly by an exoribonuclease. Probe C binds very well and will therefore inhibit degradation significantly and generate the least amount of signal in detection step (c) of the invention.

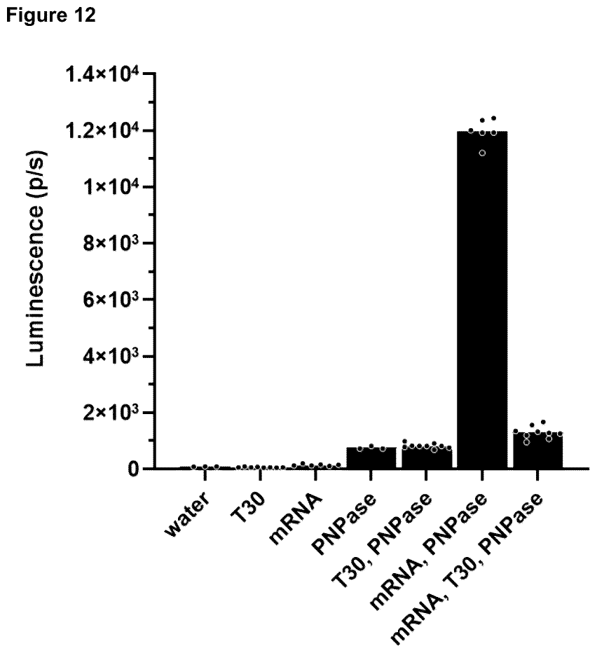

FIGS. 12 to 18 show results of Examples 9 to 15 respectively. Further details of the Figures are provided in the respective Examples.

### EXAMPLES

### Examples Using Exonuclease T

### Example 1

### Exonuclease Activity is Required to Liberate Nucleosides From mRNA

FLuc 5moU mRNA (Trilink #L-7602) was thawed at 20 ng/μl and stored on ice. Next, the mRNA was taken out of the ice and stored on the bench at room temperature for the specified time (0 h-2 h). Fifty μl of the mRNA was then diluted in 50 μl Lucipac A3 reagents (Kikkoman #60365) and measured in a Tecan Spark plate reader using flat bottom NUNC 96-well plates. In addition, a freshly thawed portion of the mRNA was diluted to 100 ng/μl and digested with 7 μl Exonuclease T (NEB #M0265) for 30 min at 25° C., followed by Exo T deactivation (for 20 min at 65° C.). Fifty μl of the resultant sample was then diluted with 50 μl Lucipac A3 reagents and further diluted in series (to a dilution of 1:10).

Exonuclease T liberates nucleoside monophosphates from ssRNA in a 3′→5′ direction (i.e., mainly the poly(A) tail is digested) and Lucipac A3 converts AMP into measurable luminescent light.

The results of this Example are shown in FIG. 1. The dotted line indicates the signal (displayed in p/s (photons per second)) obtained from 50 μl nuclease-free water with 50 μl Lucipac A3. As shown by the white bars, a small level of luminescence can be obtained with mRNA even in the absence of exonuclease T incubation. This is suspected to be due to the presence of trace AMP and/or ADP, either due to minor degradation or minor contamination of the mRNA sample. As shown by the black bars, exonuclease T incubation results in a significant increase in luminescence signal. Hence, this Example demonstrates that exonuclease activity is required to liberate nucleosides from mRNA.

### Example 2

### Different Ratios of Digested mRNA and Intact mRNA can Sensitively be Discriminated

FLuc 5moU mRNA was digested with exonuclease T and mixed with intact FLuc 5moU mRNA at different ratios (total mRNA concentration was kept constant at 10 pg/μl). Fifty μl of Lucipac A3 reagent was added to 50 μl of this mRNA mixture and luminescence was measured in a Tecan plate reader.

The results of this Example are shown in FIG. 2. For example, 1.0 indicates that the RNA mixture contained equal amounts (i.e. equal masses) of digested vs intact mRNA, whereas 0.0 indicates that the RNA mixture only contained intact. The dotted line in FIG. 2 is same as the dotted line in Example 1, i.e. it indicates the signal obtained from 50 μl nuclease-free water with 50 μl Lucipac A3.

FIG. 2 demonstrates that different magnitudes of luminescence signal result from conducting the Lucipac A3 assay on different ratios of digested mRNA and intact mRNA. Thus, this Example demonstrates that different ratios of digested mRNA and intact mRNA can sensitively be discriminated.

### Example 3

### Exonuclease T Digestion Alone Cannot be Used to Detect mRNA Degradation

FLuc 5moU mRNA (size of 1929 nt) was placed on the bench at room temperature for 1 week and compared to freshly thawed (i.e. intact) mRNA with/without Exonuclease T digestion. Fifty μl mRNA (100 pg/μl) was used and 50 μl, 25 μl, or 10 μl of Lucipac A3 reagents were added to each sample.

The results of this Example are shown in FIG. 3. A significant increase in luminescence signal is observed for the intact mRNA with exonuclease T incubation, as compared to the intact mRNA without exonuclease T incubation. This increase in luminescence signal is mainly due to the digestion of the poly(A) tails (derived from the original intact mRNA molecules) by exonuclease T. A similar increase in luminescence signal is observed for the bench mRNA with exonuclease T incubation, as compared to the bench mRNA without exonuclease T incubation. The smear on the 0.8% agarose gel (containing 1% bleach) indicates that 1 week bench mRNA is slightly degraded. However, this degradation does not result in noticeable increased luminescence.

Thus, in this example, the luminescence signal resulting from digestion by exonuclease T of poly(A) tails dominated the overall luminescence signal for the bench exo T sample, to the point that any luminescence signal resulting from digestion by exonuclease T of non-poly(A) tail ends (i.e. ends resulting from degradation of the bench mRNA sample) was not detectable.

### Example 4

### Complementary Oligonucleotides Prevent Exonuclease T Activity

Addition of Exo T to a sample of FLuc 5moU mRNA followed by 60 min incubation at 25° C. liberates AMP from ssRNA fragments. The liberated AMP is then quantified using the Lucipac A3 assay.

The results of this Example are shown in FIG. 4. Supplementing this reaction with a 5-fold molar excess of dT30 lowers the number of liberated AMP, indicating that many AMPs originate from the poly(A) tail.

### Examples Using PNPase

### Example 5

### Polynucleotide Phosphorylase (PNPase) is a 3′→5′ Exonuclease That Liberates Nucleotide Diphosphates From mRNA and That is inhibited by duplexed mRNA

FLuc 5moU mRNA was placed at room temperature for 1 week. Next, the mRNA was incubated for 2 minutes at 80° C. with and without the addition of a 5-fold molar excess of oligo-deoxythymidine (dT30). After cooling to room temperature, PNPase (Sigma #N9914) was added (final concentration of 5 ng/μl) and incubated for 60 minutes at 37° C. Next, 3 μl of this reaction (15 ng mRNA) was mixed with 25 μl Lucipac A3 reagents and luminescence was measured in a Tecan plate reader.

The results of this Example are shown in FIG. 5. Addition of dT30 inhibits liberation of ADP from the poly(A) tail.

### Example 6

### Complementary Oligonucleotides Inhibit Exonuclease-Mediated Degradation of mRNA in a Sequence-Specific Fashion

In-house poly(A)-tailed mRNA was synthetized using linearized pET22b_NEIL2-NLuc-His as template DNA in an in vitro transcription reaction (NEB #E2040S) followed by an enzymatic poly(A) tailing reaction (Cellscript #C-PAP5104H) and silica-based purification (NEB # T2040S), all according to the manufacturer's specifications. Both this non-modified and commercially acquired 5moU-modified FLuc mRNA were annealed for 2 minutes at 80° C. followed by cooling to room temperature with a 4-fold molar excess of either dT30 oligodeoxynucleotides (T) or one of three oligodeoxynucleotides (R1-R3) with a random sequence (R1:TTTACCGCAACTACACCTAACTGAGATACT (SEQ ID NO:1),

### R2:TTAGATAACCGGATACAGTGACTTTGATAG (SEQ ID NO:2),

R3:CTGCGTATGGAGGAAGGAACTTTTGCGTGT (SEQ ID NO:3)). These mRNA: oligo mixtures were then incubated with PNPase (final concentration of 10 ng/μl) in PNPase buffer (final concentration of 5 mM MgCl2, 10 mM KCl, 50 mM Tris HCl (pH 8.5), and 10 mM inorganic phosphate) for 60 min at 37° C. Six hundred ng of each reaction was then run onto a 0.8% agarose TAE gel (containing 1% bleach).

The results of this Example are shown in FIG. 6. For both the FLuc 5moU mRNA and the NEIL2-NLuc mRNA, the band in the lane containing no PNPase is approximately at the same position as the band in the corresponding lane containing both PNPase and dT30. This indicates that dT30 successfully inhibits PNPase activity. In contrast (and again for both the FLuc 5moU mRNA and the NEIL2-NLuc mRNA), the four bands in the lanes containing PNPase but lacking dT30 (either having no oligonucleotide or one of the random sequence oligonucleotides R1 to R3), are in approximately at the same position, and are lower than the other two bands (indicating higher degradation than in the other two bands). The fact that the bands in those four lanes are in the same position (for both RNA types) indicates that oligonucleotides R1 to R3 were unable to prevent mRNA degradation by PNPase. Thus, in summary, the gel image demonstrates that only dT30 inhibits PNPase activity against mRNA whereas random oligodeoxynucleotides do not.

### Example 7

### Luminescence Signal From Poly(A) Tail Phosphorolysis can be Avoided by Addition of a Large Excess of Oligonucleotides

FLuc 5moU mRNA (124 ng) was mixed with 0-500-fold molar excess of dT30 in a total of 10 μl nuclease-free water. This mixture was heated for 2 minutes at 80° C. after which it was cooled to room temperature. Next, mRNA with annealed dT30 (final concentration of 2.9 ng/μl) was mixed with PNPase (final concentration of 1.6 ng/μl) and PNPase buffer. All samples were incubated for 60 minutes at 37° C. and then centrifuged for 10 s at 8000 RPM. Using a flat bottom white NUNC 96-well plate, 5 μl of each sample was then mixed with 26 μl Lucipac A3 reagents and 25 μl nuclease-free water, and immediately imaged in a Tecan plate reader (luminescence exposure time of 2 s per well).

The results of this Example are shown in FIG. 7. The graph demonstrates that at dT30:mRNA ratio of 250-500, almost all signal coming from poly(A) phosphorylation can be suppressed. Percentage degradation may be calculated using the formula:

$$Percentage degradation = ( degradation - background ) / ( poly ( A ) - background )$$

The “background” value reflects the stable background luminescence coming from samples containing only dT30 and PNPase. The residual luminescence at complete inhibition of poly(A) phosphorolysis derives from RNA degradation fragments (“degradation”).

The “poly(A)” value and line represents the luminescence coming from the sample containing PNPase but completely lacking dT30.

At dT30: mRNA 500, the formula (degradation-background)/(poly(A)-background) suggests that the input mRNA was 11.26% degraded.

### Example 8

### The Signal Obtained From Non-poly(A) Derived RNA Fragments Correlates to RNA Degradation

FLuc 5moU mRNA in water was degraded by heating at 95° for the indicated time (0-60 min)-increased incubation time at 95 degrees will increase degradation of mRNA. One hundred nanogram mRNA was then loaded on TAE agarose gel (1% bleach). Next, 12.4 ng mRNA and 500-fold molar excess of dT30 (total volume of 10 μl) was denatured/annealed at 70° C. for 2 min in a thermocycler and cooled to room temperature. Next, mRNA only, mRNA: dT30 hybrids, and dT30 only was incubated for 60 min at 37° C. with PNPase in a 30 μl reaction (2.49 ng sample, PNPase buffer, 1.41 ng PNPase, RNase-free water). Finally, 1 μl of this mixture was mixed with 55 μl water and 5 μl Lucipac A3 reagents followed by luminescence measurement in a Tecan plate reader using white NUNCLON 96-well plates. Percentage degradation was calculated using the formula (degradation−background)/(poly(A)−background).

The results of this Example in respect of the mRNA: dT30 hybrids are displayed in FIG. 8. This Example demonstrates that the signal obtained from non-poly(A) derived RNA fragments correlates to RNA degradation. An excess of dT30 was used meaning that there was (essentially) no signal from the poly(A) tail.

### FURTHER EXAMPLES

### Example 9

### Complementary Oligonucleotides Suppress PNPase Activity

The results of this Example are displayed in FIG. 12. Intact mRNA (12.4 ng) was incubated for 2 min at 70° C. with and without 3 different batches of 500-fold molar excess oligo (T30). After cooling, these samples were then incubated at 37° C. for 60 minutes in the presence or absence of PNPase and PNPase buffer. A volume of the reaction mixture (containing 6.2 ng mRNA) was then transferred to a Nunclon 96-well plate and Lucipac A3 reagents were added. Lastly, luminescence measurement was performed 5 minutes later in a Tecan plate reader.

These results indicate that intact mRNA only generates luminescence in the presence of PNPase and that addition of oligo (T30) almost suppresses the signal generation to baseline. Moreover, limited signal variability is seen even when 3 different batches of oligo (T30) are used. These data confirm the data of FIG. 5, which used a 5-fold molar excess of T30 (the present Example is similar but uses a 500-fold molar excess of T30).

### Example 10

### RNAse R is Inhibited by Annealing Oligo (T30) to the Analyte mRNA and Both LNA or DNA Chemistries can be Used for the T30

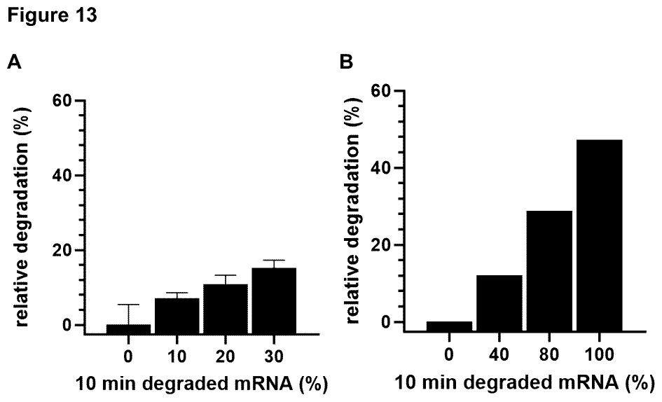

The results of this Example are displayed in FIG. 13. mRNA was heat degraded for 10 min at 95° C. After cooling, this degraded mRNA was mixed with intact mRNA in different ratios with and without addition of locked nucleic acid oligo (T30) (LNA, FIG. 13A) or deoxyribonucleic acid oligo (T30) (DNA, FIG. 13B). After annealing for 2 minutes at 60° C. followed by gradual cooling to room temperature, the samples were incubated for 60 min at 37° C. with RNAse R (Abcam #ab286929, final concentration of 0.023 U/μl) in RNAse R buffer according to the manufacturer's instructions. Five microliter of these samples was then mixed with 15 μl Lucipac A3 reagents and 40 μl dH₂O before measuring the luminescence on a Tecan plate reader. The relative degradation percentage is calculated by dividing the luminescence of annealed mRNA with the luminescence of non-annealed mRNA. The results indicate that RNAse R, a 3′-5′ exoribonuclease (Vincent and Deutscher, J Biol Chem 2006 Oct. 6; 281 (40):29769-75), is inhibited by annealing oligo (T30) to the analyte mRNA and that both LNA or DNA chemistries can be used for the T30.

### Example 11

### Higher Molar Excess of Oligos Inhibit the Digestion by Exonuclease T in a Dose-dependent Manner

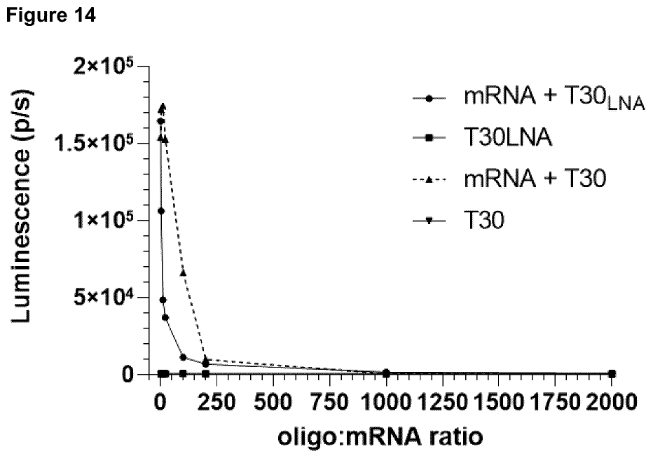

The results of this Example are displayed in FIG. 14. mRNA was heat degraded for 10 minutes at 95° C. This degraded mRNA was then annealed (2 minutes at 65° C.) to different molar quantities of oligo (T30), both of DNA chemistry (T30) and of Locked Nucleic Acid chemistry (T30LNA). These mixtures were then subjected to exonuclease T digestion (60 minutes at 25° C., enzyme inactivated by 7 min at 65° C.) followed by supplementation with Lucipac A3 reagents and luminescence readout on a Tecan plate reader. The results show that higher molar excess of oligos with different chemistries (i.e., T30 and T30LNA oligos) inhibit the digestion by exonuclease T in a dose-dependent manner.

### Example 12

### The Present Invention is Sensitive Enough to Pick Up Degradation Caused by Mild Conditions

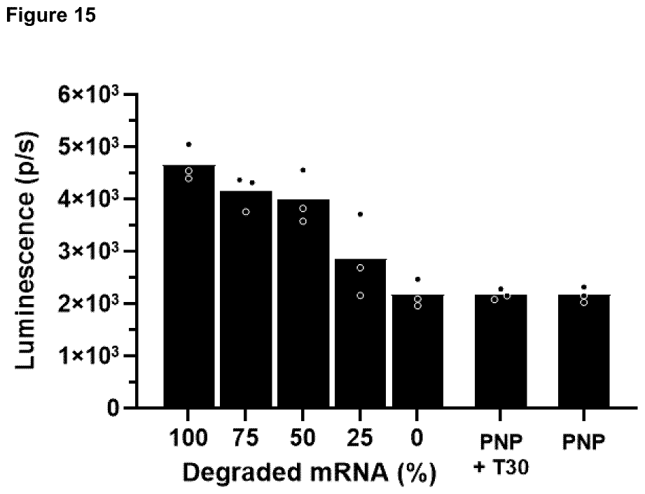

The results of this Example are displayed in FIG. 15. mRNA was heat degraded for 10 minutes at 70° C., annealed (10 minutes at room temperature) to a 1000-fold molar excess oligo (T30), mixed in different ratios with intact mRNA, and subjected to PNPase digestion (60 minutes at 37° C.) followed by supplementation with Lucipac A3 reagents and luminescence readout on a Tecan plate reader. Even at these very mild heat degradation conditions, the results show increased luminescence signal when more degraded mRNA is present in the sample. Under these conditions, such differences could not be detected on capillary gel electrophoresis using the method of Raffaele et al. Electrophoresis 2022 May; 43 (9-10): 1101-1106.

### Example 13

### Luminescence in PNPase-based Assay Using Various Excesses of T30 and Mildly Degrading Conditions

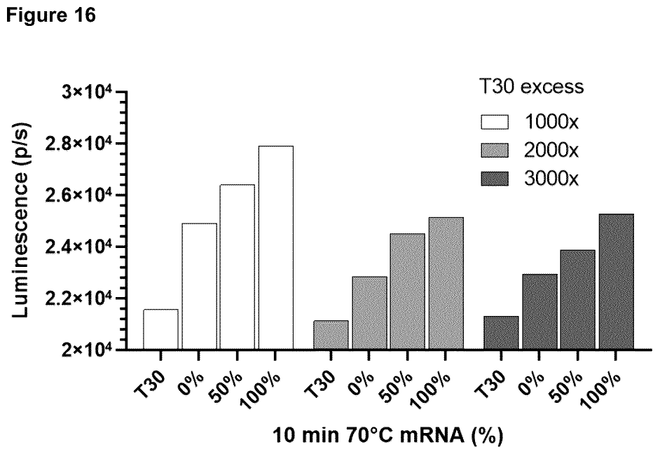

The results of this Example are displayed in FIG. 16. mRNA was heat degraded for 10 minutes at 70° C. and mixed with intact mRNA at different ratios (a total of 12 ng input mRNA per sample). Next, a 1000-fold, 2000-fold, or 3000-fold molar excess of oligo (T30) was added to the mRNA samples and annealed for 10 min at room temperature. After brief centrifugation, PNPase was added in PNPase buffer and incubated at 37° C. for 10 minutes followed by addition of Lucipac A3 reagents and measurement of luminescence with a Tecan plate reader. These results confirm (a) the difference in luminescence output between intact mRNA and mRNA that was mildly heat degraded at 70° C., (b) that differences in luminescence can be obtained by digesting the mRNA samples for only 10 minutes, (c) that no significant signal improvement can be seen when adding >1000-fold T30.

### Example 14

### The assay Outputs Higher Luminescence When More Untailed mRNA Fragments are Present and Available for Digestion

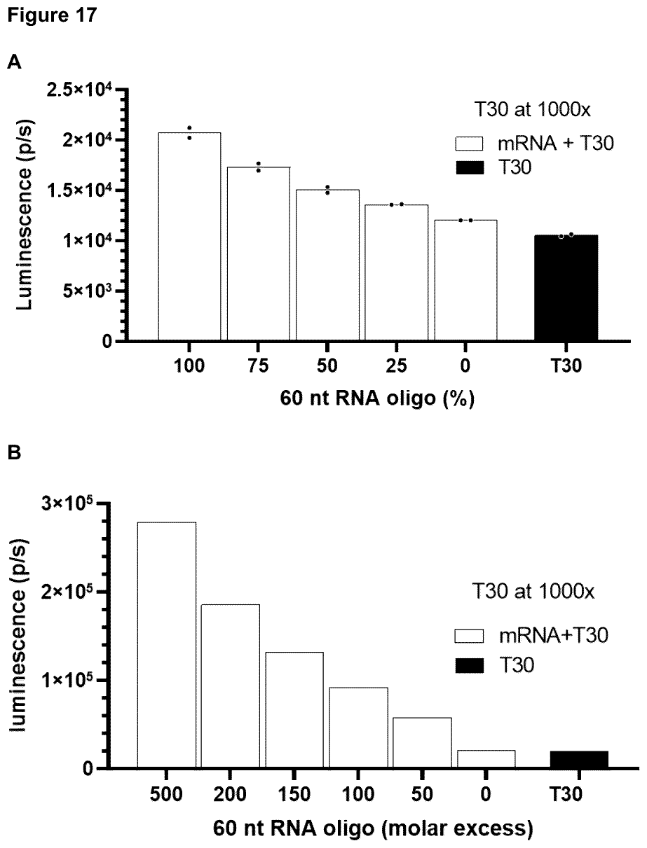

The results of this Example are displayed in FIG. 17. FIG. 17A: Twelve nanogram mRNA was mixed with a 60 nt RNA oligo at different molar ratios in the presence of a 1000-fold molar excess oligo (T30) which were allowed to anneal to the mRNA over 10 minutes at room temperature. Next, PNPase in PNPase buffer was added to each mRNA sample and incubated for 5 minutes at 37° C. followed by addition of Lucipac A3 reagents and luminescence measurement on a Tecan plate reader. FIG. 17B: various molar excesses of RNA oligos were mixed with intact mRNA and treated in the same way as the RNA in the left figure. Taken together, these experiments imitate the increasing number of RNA fragments that arise due to mRNA degradation. The results demonstrate that, like heat degraded mRNA, the assay outputs higher luminescence when more untailed mRNA fragments are present.

### Example 15

### The 5′→3′ Exoribonuclease XRN-1 can be Used to Liberate AMP From Decapped mRNA

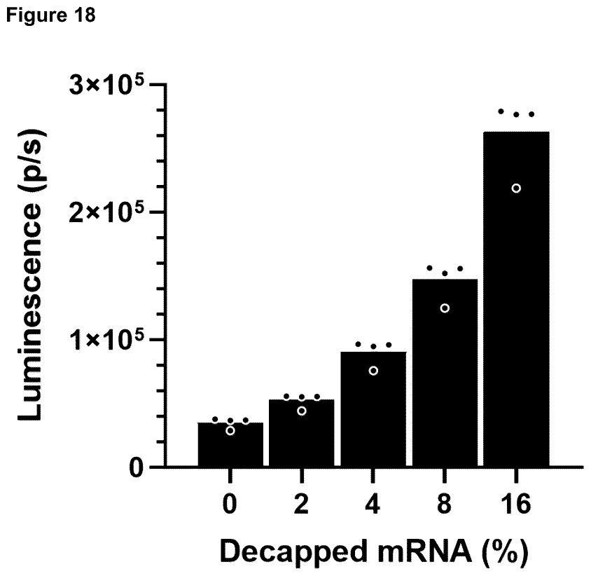

The results of this Example are displayed in FIG. 18. mRNA was decapped using DCP-1 thereby generating 5′ monophosphate mRNA. This decapped mRNA was mixed with capped mRNA in different ratios (0-16%). Next, an oligonucleotide complementary to the 5′ UTR (GGGTTCTCTCTGAGTCTGT (SEQ ID NO:4)) was annealed (2 minutes at 60° C.) to the analyte mRNA to prevent excess signal generation. This mixture was then incubated with 5′-3′ exoribonuclease 1 (XRN-1) for 10 minutes at 37° C. according to the manufacturer's specifications (NEB #M0338S). Lastly, Lucipac A3 reagents were added and luminescence was measured on a Tecan plate reader. These results demonstrate that XRN-1 can be used to liberate AMP from uncapped mRNA. In turn, this AMP can be converted into measurable luminescence and related to the percentage of decapped mRNA in a sample.

1. A method of analysing ssRNA in a sample, said method comprising:
(a) contacting the sample with a probe, wherein the probe binds to a target region of ssRNA in the sample;
(b) incubating the sample with a single-strand specific exoribonuclease; and
(c) detecting nucleotides and/or nucleosides generated by step (b), wherein the nucleotides and/or nucleosides are derived from one or more RNA regions located outside the target region of ssRNA in the sample.

2. The method of claim 1, wherein the nucleotides and/or nucleosides are adenosine, adenosine monophosphate, adenosine diphosphate or a combination thereof, preferably adenosine monophosphate, adenosine diphosphate or a combination thereof.

3. The method of claim 1 or claim 2, wherein the detecting step relies on a light output.

4. The method of any one of the preceding claims, wherein the detecting step uses an enzyme-reagent mixture, preferably wherein the enzyme-reagent mixture comprises luciferase, more preferably also pyruvate orthophosphate dikinase and/or pyruvate kinase.

5. The method of any one of the preceding claims, wherein the probe is an oligonucleotide probe, preferably a DNA or RNA oligonucleotide probe, more preferably a DNA oligonucleotide probe.

6. The method claim 5, wherein the oligonucleotide has a length of 15 to 100 nucleotides, preferably 20 to 60 nucleotides.

7. The method of any one of claims 1 to 4, wherein the probe is an RNA binding protein.

8. The method of any one of the preceding claims, wherein the exoribonuclease is a 3′→5′ exoribonuclease.

9. The method of claim 8, wherein the ssRNA comprises a poly(A) tail and the target region of ssRNA is said poly(A) tail.

10. The method of claim 8, wherein the ssRNA comprises a poly(A) tail and the target region of ssRNA is immediately upstream of said poly(A) tail or spans the 5′ end of the poly(A) tail.

11. The method of any one of claims 1 to 7, wherein the exoribonuclease is a 5′→3′ exoribonuclease.

12. The method of claim 11, wherein the target region of ssRNA is located at or near the 5′ end of the ssRNA.

13. The method of any one of the preceding claims, wherein the exoribonuclease is a hydrolytic exoribonuclease or a phosphorolytic exoribonuclease.

14. The method of any one of the preceding claims, comprising performing a further method on the same sample material in which step (a) as defined in any one of the preceding claims is not performed but steps (b) and (c) as defined in any one of the preceding claims are performed.

15. The method of any one of the preceding claims, wherein the method is for measuring the binding efficiency of the probe to the ssRNA.

16. The method of any one of the preceding claims, wherein the method is for analysing secondary structures within the ssRNA, preferably stem loop or hairpin formation in the ssRNA.

17. The method of any one of the preceding claims wherein the ssRNA comprises one or more modified nucleotides, preferably wherein the one or more modified nucleotides comprises N1-methylpseudouridine.

18. A kit for performing a method of ssRNA analysis as claimed in any one of the preceding claims, said kit comprising the probe as defined in any one of the preceding claims and the exoribonuclease as defined in any one of the preceding claims, optionally together with an enzyme-reagent mixture for detecting nucleotides and/or nucleosides.
